(19) United States
(12) Patent Application Publication
(10) Pub. No.: US 2010/0010290 A1
(43) Pub. Date: Jan. 14, 2010

(54) Smooth muscle implant for managing a medical condition

(75) Inventors: Anthony Clyde Neason Stephens, New South Wales (AU); John Furness, Victoria (AU); George David Scarcliffe Hirst, Victoria (AU)

Correspondence Address: JONES DAY, 222 EAST 41ST ST, NEW YORK, NY, 10017, US

(73) Assignee: Continence Control Systems International Pty Ltd., Chatswood, New South Wales (AU)

(21) Appl. No.: 11/991,258

(22) PCT Filed: Sep. 4, 2006
(86) PCT No.: PCT/AU2006/001301

§ 371 Date: Aug. 26, 2009

(30) Foreign Application Priority Data
Sep. 2, 2005 (AU) 2005904830
Oct. 14, 2005 (AU) 2005905672
Jan. 16, 2006 (AU) 2006900210

(51) Int. Cl. A61F 2/04; A61L 27/54; A61N 1/36

(52) U.S. Cl. 600/30; 424/423; 607/41

(57) ABSTRACT

The present invention relates to an implant for managing a medical condition. In particular, it relates to medical treatments which employ smooth muscle implant and discloses a method of augmenting a smooth muscle implant for management of a human medical condition. Augmentation may be by a number of methods. It may be by utilising neurotrophic or trophic factors to assist growth of the smooth muscle tissue or nerve growth within the smooth muscle tissue, or revascularisation of the smooth muscle tissue. It may be by utilising tissue engineering to grow smooth muscle tissue. It may be by adding proliferative smooth muscle cells and/or smooth muscle stem cells for the smooth muscle.

### RELATED APPLICATIONS

U.S. Pat. No. 6,659,936 issued on 9 Dec. 2003, International Patent Application PCT/AU00/00925 filed on 4 Aug. 2000, Australian Provisional Application AU PQ2026, filed on 4 Aug. 1999, relate to the control of continence.

International Patent Application PCT/AU2006/000258 filed on 2 Mar. 2006, Australian Provisional Application AU2005/00957 filed on 2 Mar. 2005, relate to improvements to managing incontinence.

International Patent Application PCT/AU2005/001698 filed on 8 Nov. 2005, Australian Provisional Application AU2004906393, filed on 8 Nov. 2004, relate to an implantable electrode arrangement.

Australian Provisional Application no. 2005904830 filed on 2 Sep. 2005 relates to an implant for managing a medical condition.

Australian Provisional Application no. 2006900210 filed on 16 Jan. 2006 relates to a stimulator for applying stimulation to control a bodily function.

Australian Provisional Application no. 2005905672 filed on 14 Oct. 2005 relates to a method and apparatus for treating a heart condition.

Each one of the above documents are incorporated herein by reference in their entirety.

### FIELD OF THE INVENTION

The present invention relates to an implant for managing a medical condition, and particularly, but not exclusively, to medical treatments employing a smooth muscle implant and a method of facilitating a smooth muscle implant for management of a human medical condition.

### BACKGROUND OF THE INVENTION

It is known to use innervated smooth muscle implants in order to treat some medical conditions. International Patent Application PCT/AU00/00925 discloses a method and apparatus for managing urinary incontinence in humans. A “neosphincter” is formed from smooth muscle tissue taken from elsewhere in the patient's body, and wrapping the smooth muscle tissue around the urethra. An implantable stimulator provides an electrical signal to the neosphincter by way of an electrode that delivers the electrical signal. The electrical signal stimulates the neosphincter to maintain tension in the muscle around the urethra to prevent emptying of the bladder until the user wishes to urinate. The stimulator may provide a further electrical signal (or stop providing signals) to allow the neosphincter to relax and so enable the individual to urinate.

Where smooth muscle is taken from another part of a patient's body in order to prepare an implant for treatment of a condition, the smooth muscle so-obtained may not be in optimum condition. For example, trauma may have occurred resulting in cell damage (particularly in the areas of the smooth muscle proximate the surgical incision sites). As another example, the smooth muscle may have reduced contractile smooth muscle due to aging of the patient or hormonal status (e.g. post-menopause). Additionally, the configuration of the smooth muscle from the removal site may not be optimal for operation in the required application. A further problem is that there may be fibrous tissue arising from an earlier surgical procedure, which may mean smooth muscle content of the proposed graft material is less dense than ideal. Yet a further problem may be that the intended location of the smooth muscle implant is not easily re-vascularised (e.g. there are fibres adhesions from earlier surgical procedure(s)), so that there is a risk that the smooth muscle implant will not receive adequate re-vascularisation to ensure good tissue viability in subsequent re-innervation).

### SUMMARY OF THE INVENTION

In accordance with a first aspect, the present invention provides a method of facilitating a smooth muscle implant, comprising the step of augmenting smooth muscle intended for an implant.

In an embodiment, the smooth muscle implant is already implanted when the step of augmentation is carried out. In another embodiment, the step of augmenting the smooth muscle is carried out on smooth muscle that is not implanted, to create an augmented smooth muscle device that subsequently can be implanted.

In an embodiment, the step of augmenting comprises the step of adding smooth muscle cells in their proliferative state to the smooth muscle.

The proliferative smooth muscle cells may have previously been harvested from the patient, or histocompatible cells may be provided from another source.

The smooth muscle cells may be added by injecting them into or proximate to the smooth muscle intended for implant or injected into or added to the smooth muscle following implant.

In an embodiment, the step of augmenting comprises the step of adding smooth muscle stem cells to the smooth muscle. Addition cells may be added by way of injection. The smooth muscle stem cells may be added to the smooth muscle intended for implant or to the smooth muscle following implant.

Smooth muscle stem cells include, without restriction of meaning, stem cells that are derived from adult or embryonic tissue that are capable of developing into smooth muscle cells either by manipulation in tissue culture or following placement into a patient.

The addition of the proliferative or stem cells may be at a plurality of sites within the smooth muscle.

An advantage of proliferative and smooth muscle stem cells is that they will grow relatively quickly and augment by strengthening the smooth muscle implant. The smooth muscle implant may provide a scaffold for proliferative and/or smooth muscle stem cell growth alignment.

Note that, in an embodiment, both smooth muscle stem cells and proliferative smooth muscle cells may be used for augmentation.

The addition of the proliferative or smooth muscle stem cells may augment by increase in speed of growth of the implant or intended implant and reinforcement of the implant or intended implant.

It is generally desirable for a smooth muscle implant that it be innervated. This may particularly be the case where the smooth muscle implant is required to have a contractile function (such as with a sphincter) and where electrical stimulation is applied to the innervation of the smooth muscle in order for contraction to occur.

In an embodiment, the step of augmenting the smooth muscle comprises the step of adding a neurotrophic factor or neurotrophic factors in order to augment by promoting nerve growth. The neurotrophic factor may be a nerve growth factor.

The neurotrophic factor(s) may be utilized to stimulate growth of specific classes of nerve fibers. This may result in overcoming problems of growth inhibition by scar tissue and the slower rates of ingrowth in elderly patients. One specific class is noradrenergic nerve fibers.

The neurotrophic factors may be selected to promote ingrowth of noradrenergic fibers over cholinergic and sensory nerve fibers.

The neurotrophic factors may augment by causing ingrowth of nerve fibers at the implant site into the implant (i.e. existing patient nerve fibers).

The neurotrophic factors may be added to the smooth muscle implant prior to implantation to create an augmented smooth muscle device for implantation.

In an embodiment, the step of adding the neurotrophic factors comprises the step of implanting a slow release delivery mechanism for delivering the growth factor, proximate to the implanted smooth muscle site.

The method may include the step of providing a slow release delivery system for providing the neurotrophic factors to the smooth muscle to be augmented.

In an embodiment, the step of augmenting the smooth muscle comprises the step of adding trophic factor or trophic factors in order to promote the growth of healthy smooth muscle tissue.

In an embodiment, the smooth muscle tissue may be augmented before implanting.

In an alternative embodiment, the trophic factor(s) may be delivered by a device to the implanted smooth muscle tissue.

In an embodiment, the smooth muscle implant augmented in accordance with one or more of the above methods may advantageously ameliorate or overcome the problems which may have resulted from, for example, smooth muscle being taken from another part of a patient's body or a donor's body, resulting in trauma, and may also or alternatively overcome the disadvantages of implanting the smooth muscle at a site which does not readily facilitate re-innervations or re-vascularisation. In an embodiment, augmentation of the smooth muscle may be able to alter the configuration to make it more optimal for operation in the required application. In an embodiment, the density of the smooth muscle may be increased by augmentation to facilitate operation in the application.

In one embodiment, the step of augmenting the smooth muscle may comprise the step of preparing a partial or an entire smooth muscle device for implant by utilising tissue modelling. In this embodiment, an in vitro culture may be used to tissue engineer a smooth muscle of the desired configuration.

This may have the advantage of avoiding potential problems with injured smooth muscle cells removed from the patient. It may avoid inflammatory reactions.

The tissue engineering may occur on a matrix (e.g. a collagen matrix providing a connective tissue substrate, or a synthetic matrix). In one embodiment, tension may be applied to the matrix in order to orientate the growth of the smooth muscle cells in the desired direction.

Note that, with this embodiment, other methods of augmentation may be used to assist with the tissue modelling e.g. additional proliferative and/or smooth muscle stem cells, and/or growth factors.

One or more of the augmentation methods discussed above may be used in combination to facilitate an innervated smooth muscle implant.

The method of this aspect of this invention may be used to prepare a smooth muscle implant operating as a sphincter. In order to treat a condition requiring a sphincter, the sphincter may be activated by electrical signals provided by an implanted stimulator. For some conditions the stimulator may be under a user's control.

The sphincter may be an augmented smooth muscle sphincter for the management of urinary incontinence, and may be implanted and stimulated in the same way as described in the PCT Application PCT/AU00/00925.

In accordance with a second aspect, the present invention provides a smooth muscle implant augmented in accordance with the method of the first aspect of the invention.

In an embodiment, the smooth muscle implant is arranged to serve a contractile tissue function in a patient's body.

The smooth muscle implant in an embodiment is a smooth muscle sphincter. In an embodiment, the smooth muscle sphincter is an innervated smooth muscle sphincter.

In an embodiment, the smooth muscle sphincter is a sphincter for management of urinary incontinence and is arranged for positioning about the urethra of a patient.

In an embodiment, the smooth muscle sphincter is a sphincter for management of fecal incontinence and is arranged for positioning about the colon of a patient in the anal area.

In accordance with a third aspect, the present invention provides a system for managing a patient condition, comprising an implantable stimulator device and a smooth muscle implant in accordance with the second aspect of the invention, the implantable stimulator comprising a signal generator for stimulating the implanted augmented smooth muscle.

In an embodiment, the smooth muscle implant is arranged to serve a contractile tissue function in a patient's body.

In an embodiment, the implanted augmented smooth muscle is a smooth muscle sphincter.

In an embodiment, the smooth muscle sphincter is a sphincter for the treatment of urinary incontinence.

In other embodiments, the smooth muscle sphincter is a sphincter for the treatment of fecal incontinence.

In an embodiment, the system further comprises a controller which is operable by a user to control the implantable stimulator device.

In an embodiment, the system further comprises a programmer, which is operable by a clinician to programme parameters of the implantable stimulator.

In an embodiment, the system comprises an implantable delivery device, arranged to deliver neurotrophic and/or trophic factor(s) to promote nerve growth and/or tissue growth in the smooth muscle implant. In an embodiment, the implantable delivery device is mounted by an electrode arranged to apply a signal from the stimulator to the smooth muscle implant. In an embodiment, the delivery device is part of the electrode.

In an embodiment, the delivery device is mounted with a supporting material placed in the vicinity of the smooth muscle implant. In an embodiment, the supporting material is a supporting mesh. The mesh may be impregnated or coated with neurotrophic factor(s) and/or trophic factor (s) so as to be the delivery device, or may mount a delivery device with a neurotrophic factor(s). Alternatively the supporting material may be made of a suitable biocompatible material such as silicone rubber that has the neurotrophic or trophic factors impregnated in it. Alternatively, the supporting material may be a suitable biocompatible silicone rubber which includes embedded mesh that provides greater tear resistance to the silicone, however in addition, the mesh within the silicone may be coated with the neurotrophic or trophic factors. Alternatively the supporting material of the delivery device may include a polymer matrix that is placed within the supporting material that incorporates the neurotrophic or trophic factors. Alternatively the supporting material that includes a polymer matrix that is placed within it can also incorporate methods intended to more precisely control the rate of release of the neurotrophic and/or trophic substances, such as polymer nanotubes.

In accordance with a fourth aspect, the present invention provides a delivery device for delivering a neurotrophic and/or trophic factor to a smooth muscle implant in order to augment the smooth muscle implant.

In an embodiment, the delivery device is an electrode intended for stimulation of the smooth muscle implant. In an embodiment, the delivery device is mounted by an electrode intended for stimulation of the smooth muscle implant.

In an embodiment, the delivery device is a support material for supporting tissue in the body, organs in the body, or the smooth muscle implant. In an embodiment, the delivery device is mounted by support material for supporting tissue, organs or the smooth muscle implant.

In an embodiment, the delivery device is arranged to deliver the factor(s) to a smooth muscle implant outside the body, to produce a smooth muscle implant device intended for implant in the body.

In accordance with a fifth aspect, the present invention provides a system including a smooth muscle implant and a delivery device in accordance with the fourth aspect of the invention.

In accordance with a sixth aspect, the present invention provides a method of facilitating a smooth muscle implant, comprising the step of preparing a partial or entire smooth muscle device for implant by utilising tissue modelling.

In an embodiment, the step of tissue modelling utilises an in vitro culture to tissue engineer the smooth muscle implant of the desired configuration.

In accordance with a seventh aspect, the present invention provides a smooth muscle implant prepared in accordance with the method of the sixth aspect of the invention.

### BRIEF DESCRIPTION OF THE DRAWINGS

Features and advantages of the present invention will become apparent from the following description of embodiments thereof, by way of example only, with reference to the accompanying drawings, in which;

FIG. 1 is a schematic diagram showing smooth muscle implanted or intended for an implant, being augmented by the addition of further cells in accordance with an embodiment of the present invention; and

FIG. 2 shows a system including an implanted neosphincter and stimulator, the neosphincter being augmented in accordance with an embodiment of the present invention;

FIG. 3 is a block diagram of a stimulator of the system of FIG. 2;

FIG. 4 is a block diagram of an apparatus including the stimulator of FIG. 3;

FIG. 5 is a block diagram of a further apparatus including the stimulator of FIG. 3;

FIG. 6 is a cross-sectional diagram of the colorectal anatomy showing an implanted stimulator device and sphincter in accordance with an embodiment of the present invention;

FIGS. 7, 8 and 9 are exploded perspective, plan and side views, respectively, of an electrode arrangement for stimulating a sphincter in accordance with an embodiment of the present invention;

FIGS. 10, 11, 12, and 13 are perspective, plan, side section, plan section and detailed views of a shroud component of the electrode arrangement of FIG. 6;

FIGS. 15, 16, 17, 18, 19 are perspective, rear, plan section, side section and plan views of a cover component of the electrode arrangement of FIG. 6;

FIG. 20 is a perspective view from above and one side of an electrode arrangement incorporating device in accordance with an embodiment of the present invention;

FIG. 21 is a exploded perspective view of a electrode arrangement in accordance with an embodiment of the present invention;

FIG. 22 is a perspective view from above and one side of an electrode arrangement in accordance with an embodiment of the present invention;

FIG. 23 is a perspective view from above and one side of an electrode arrangement in accordance with a further embodiment of the present invention;

FIG. 24 is a perspective view from above and one side of a further embodiment of an electrode arrangement in accordance with the present invention; and

FIG. 25 is a diagram of a system for treating a heart condition, incorporating an embodiment of the present invention.

### DETAILED DESCRIPTION OF EMBODIMENTS

In accordance with embodiments of the present invention, smooth muscle implanted or intended for implantation is augmented to facilitate performance. In cases where smooth muscle has been removed from a surgical site and is damaged, the augmentation may serve to overcome problems-caused by the damage. In cases where the site of implantation of the smooth muscle is not ideal to promote vascularisation and nerve growth, the augmentation may overcome these problems.

By the term “augmentation” this is meant that the smooth muscle implant or intended for implant is altered or built in a way so that it is improved over a smooth muscle obtained by the standard procedure (usually by transplanting from another part of the body). It may be improved because it results in repairing of damage that may otherwise not be repaired, or repaired slowly, more rapid innervation, or other improved effect. In one example, augmentation means that the smooth muscle implant is altered in a way so that it can generate a greater biological effect for the same intensity of stimulation than if it were not altered. This can manifest itself in several ways of facilitated performance:

(a) For some smooth muscle that is harvested at surgery, the augmentation may result in greater biological effect for the same intensity of simulation. For example, in a sphincter application, this may mean greater forced closure collapse of a lumen for a given stimulus intensity.

(b) The smooth muscle that is scarred or in a less than ideal state due to previous surgery or trauma and that may have increased fibrous tissue, adhesions etc, the augmentation may restore the smooth muscle implant to untraumatised state.

(c) For a smooth muscle implant created by tissue engineering, the augmentation can result in a greater biological effect than can be achieved by simply laying down the smooth muscle.

In terms of effect on clinical function, in the short term, augmentation may speed revascularisation and reinnervation of the smooth muscle so that it will respond to electrical stimulation sooner, so the system can be switched on sooner after surgery. In the longer term, augmentation may result in achieving the same biological effect with less stimulation intensity, prolonging battery life. It may also result in greater stability of biological effect so that the same stimulation parameters can be used following switch-on, reducing the need for the patient to return to the clinic for re-programming.

Augmentation may be achieved by a number of methods.

1. Smooth muscle may be “reinforced” by the addition of proliferative or smooth muscle stem cells. Cells in their proliferative state may be harvested from a patient requiring an implant. Smooth muscle stem cells from the patient may be utilised. Alternatively, stem or proliferative histocompatible cells from another source may be utilised.

The cells may be prepared in culture and injected into the smooth muscle at one or multiple sites.

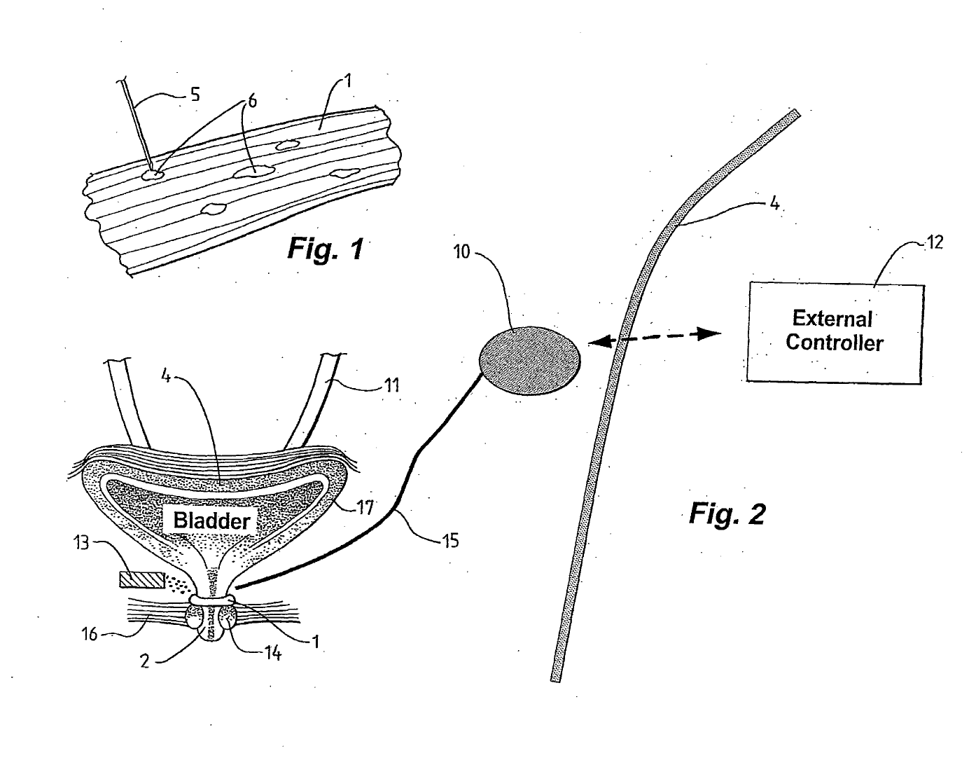

The smooth muscle intended for implant or already implanted may have been surgically removed from another site on the patient. In this case, there may have been some trauma to the smooth muscle, or the smooth muscle cells may otherwise be insufficient to perform the required function (which may be a sphincter function). FIG. 1 illustrates injection by needle 5 of additional smooth muscle cells 6 at multiple sites on a smooth muscle implant or intended implant 1.

The smooth muscle 1 provides a scaffold for alignment and growth of the injected cells 6.

In one embodiment, the smooth muscle 1 is an implant or a device intended as an implant as a sphincter for managing urinary incontinence.

In other embodiments, the smooth muscle is not intended as a sphincter, but may be used in any other application where contractile tissue is utilised, for example, to play a “mechanical” role within the body.

2. Smooth muscle may be treated with neurotrophic factors, such as growth factors, such as NGF (nerve growth factor) or GDNF (glial derived neurotrophic factor) (or any other neurotrophic factor) in order to increase the degree to which nerve fibers grow into the transplant. Where innervation is required for functioning of the implant, as is the case in most sphincter implants, for example, this technique may be useful in ensuring correct operation of the implant.

3. The smooth muscle may be treated with trophic (growth) factors to facilitate the growth of healthy smooth muscle tissue and re-vascularisation.

A delivery device may be utilised to deliver trophic and/or neurotrophic factors to the smooth muscle intended as an implant, or to smooth muscle already implanted.

In an embodiment, an electrode used to stimulate and control the smooth muscle may incorporate trophic factor(s) to facilitate the growth of healthy smooth muscle tissue in the vicinity of the conductive surfaces of the electrode. The electrode may also incorporate means for delivering neurotrophic factor(s) for stimulating growth of nerve fibers.

In an embodiment, neurotrophic factors are utilised to stimulate growth of nerve fibers already existing at the implant site, e.g. noradrenergic neurons in the pelvic area of a patient being treated for urinary incontinence.

FIG. 2 shows a system for treating urinary incontinence which includes an implanted smooth muscle neosphincter 1 positioned about the external urethra 2 and arranged to be stimulated by an implanted stimulator device 10 to prevent or reduce leakage of urine from the bladder 11. A lead 15 extends from the stimulator 10 to an electrode on or in the neosphincter 1. An external controller 12 is arranged to provide control signals and for operation by the patient in order to cause the stimulator 10 to maintain signals to the neosphincter 1 to maintain bladder 4 tone or turn off the signals so that the patient can urinate. A similar system is described in PCT/AU00/00925.

Proximate to the neosphincter 1, is a device 13 in the form of a slow release implant arranged to provide a slow release of growth factor(s), (such as NGF, or vascular endothelial growth factor (VEGF)).

The device 13 is positioned proximate to the neosphincter 1 within the pelvic anatomy of the patient. It is positioned so that the growth factor stimulates nerves around the bladder (stemming from ganglia near the bladder) to grow into the implanted sphincter 1. This will strengthen the innervation of the neosphincter 1 and lead to improved performance, and establishment of functional innervation in a shorter time period than would otherwise be the case.

The addition of neurotrophic and/or other trophic factor(s) may also overcome problems of ingrowth inhibition by scar tissue and the slower rates of growth in elderly patients. The trophic factors may be selective for specific sub-groups of nerve fibers, e.g., noradrenergic over cholinergic and sensory nerve fibers.

Other features of the pelvic anatomy are illustrated in FIG. 2, including the ureters 11, the External Urethral Sphincter (EUS) 14, the pelvic floor 16, the detruser smooth muscle 17.

The stimulator 10 may be implanted in any surgically convenient position, but is preferably implanted between the abdominal muscles and the skin (represented by the line designated by reference numeral 4).

The diagram of FIG. 2 illustrates the female anatomy. A system including a stimulator 10, neosphincter 1 and delivery device 13 may also be implanted in the male anatomy. The invention is not limited to use with females. Note that the invention may also be utilised with animals and is not limited to humans.

4. In vitro tissue modelling to create a preformed smooth muscle contractile device, such as a sphincter, for example, using proliferative or smooth muscle stem cells extracted from a patient or from a histocompatible source.

This embodiment is to build an entirely constructed smooth muscle and avoid the need to take smooth muscle from part of the patient's body for transplant as a sphincter. Instead, proliferative or stem cells are extracted and grown on a collagen or synthetic scaffold (as is known in tissue modelling). In order to ensure that the smooth muscle cells are correctly aligned, tension may be applied in the desired growth direction to the supporting scaffold. Growth factors may be used to promote smooth muscle growth and differentiation and/or to promote nerve fiber growth when the smooth muscle is implanted.

Artificial scaffolds may be used to tissue engineer. This means that the smooth muscle can be grown in a variety of configurations.

Techniques 1, 2 and 4, may be used in any combination to facilitate a smooth muscle implant.

The augmented smooth muscle implant may be used to treat any condition where it would be useful. In one embodiment, it may be used to treat urinary incontinence by the preparation of a smooth muscle sphincter. It may also be used to treat esophageal reflux and/or fecal incontinence. It may also be useful in treating cardiac conditions.

A more detailed description will now be given of the stimulator 10 and associated components of the system including the external controller 12 and a programmer.

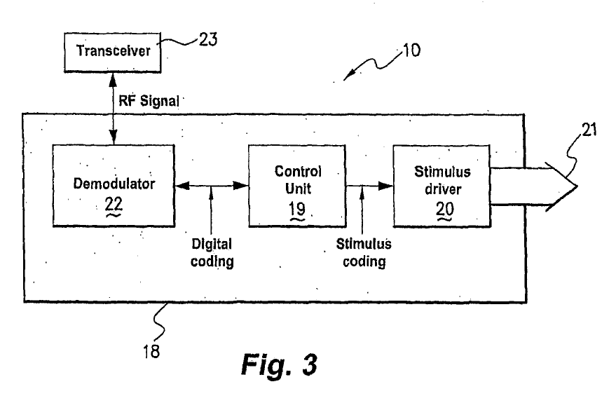

The stimulator 10 is shown in more detail in FIG. 3. In this embodiment, a signal generator arranged to provide the electrical signal for stimulation of the smooth muscle includes a control unit 19 and stimulus driver 20. The control unit 19 encodes the stimulus and provides a signal to the stimulus driver 20 which provides the stimulation signal at output 21. The output 21 may output to one or more conductors 15 as required and to one or more electrodes. The control unit 19 may be arranged to control the stimulus driver 20 to provide a plurality of stimulation signals e.g. one or more stimulation signals to contract the smooth muscle implant 1.

In this embodiment, the control unit 19 and stimulus driver 20 form, together with a demodulator/modulator 22, a processing unit 18 for generating the stimulation signal(s) at output 21.

The demodulator/modulator 22 is arranged to demodulate a signal received by transceiver 23. An external control unit 12 and external programmer unit (both to be described later) are able to communicate via the transceiver 23 with the processing unit 18 in order to control application of stimuli and/or vary the stimuli. In addition, as described in more detail later, the processing unit 18 may transmit, via control unit 19, demodulator 22 and transceiver 23, signals to the control unit or programmer unit. The transmitted signals may deliver telemetry information indicative of parameters of the stimulator, for the purposes of calibration and control.

The entire stimulator 10 (including components 18 and 23), is enclosed in a housing which includes a casing made from a bio-compatible material, such as titanium, silicone rubber or other known inert materials. The frequency of the RF signal for transmission and reception by the transceiver 23 may depend on the material of the casing of the stimulator.

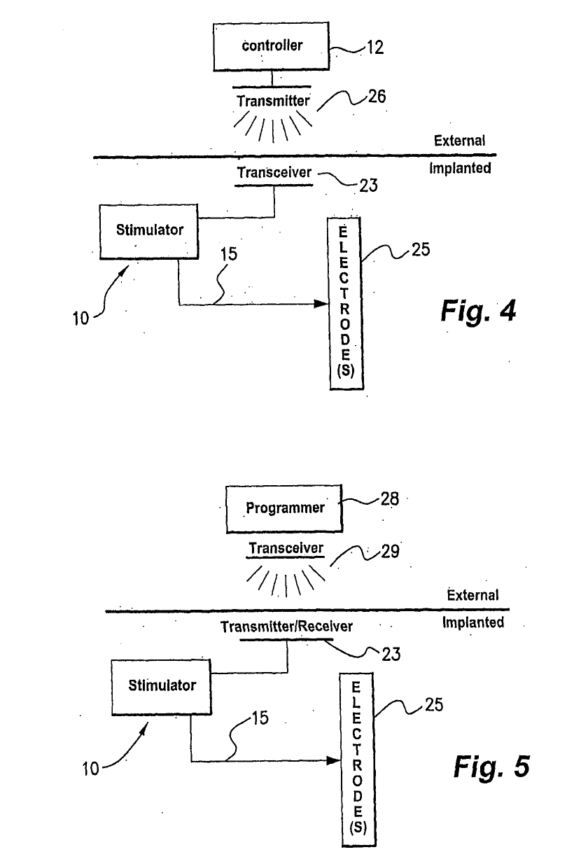

FIG. 4 shows a system incorporating the stimulator 10 of FIG. 3. The electrode(s) 25 is shown schematically together with cable(s) 15.

The apparatus also comprises an external controller 12 which includes a transmitter 26. The controller 12 is intended for operation by a patient with the stimulator implanted, for control of the stimulator 10.

The controller 12 includes an actuator (such as a button, not shown) operable by the patient to selectively send signals to the implanted stimulator 10, for control of the stimulation signals being sent to the electrodes 25. In this embodiment, the stimulator is “fail safe”. Unless a signal is received from the controller 12, the stimulator produces a signal which maintains tone in the smooth muscle implant 1. In the embodiment where the smooth muscle implant 1 is a smooth muscle sphincter for controlling urinary incontinence, when the patient wishes to urinate, they actuate the controller 12 to send, via the transmitter 26, a signal to the stimulator 10. In response to receiving the signal, the control unit 19 operates to turn the stimulating signal off causing the sphincter to relax and allow the patient to urinate.

The controller 12 may also be arranged to provide a further signal under patient control, once the patient has finished urinating, the further signal causing stimulator 10 to resume providing the stimulation signals to the electrode(s) 25.

In “fail safe” mode, if the further signal is not produced, the stimulator will resume providing the stimulation signal to the electrodes 25 after a pre-determined period of time.

The stimulation signal 21 provided to contract the smooth muscle sphincter 1 is selected so as to provide a substantially continuous tone in the sphincter 1. A generally rectangular and symmetrically biphasic pulse may be suitable for this. The signal has a substantially constant current less than or equal to 50 mA, 15 mA, 10 mA, or 5 mA, and in some preferred embodiments may be in the order of 4 mA, 8 mA, 12 mA, or 15 mA. Stimulation pulse frequency provided to sphincter 1 is in the range of 0.1 Hz to 5 Hz, 0.2 Hz to 4.0 Hz. 0.25 Hz to 3.0 Hz, 1 Hz to 3.0 Hz, 1.5 Hz to 3 Hz, 1.75 Hz to 2.5 Hz, or a 0.25 Hz to 2.25 Hz, and in one embodiment, is 1 Hz, 2 Hz, 2.5 Hz or 3 Hz. Stimulation phase width of each phase is in the range of 0.05 ms to 2.0 ms, 0.1 ms to 1.5 ms, 0.2 ma to 1 ms, 0.25 ms to 0.75 ms, and in one embodiment is 0.2 ms, 0.4 ms, 0.5 ms or 1 ms. The stimulator is current regulated, and accordingly the stimulation voltage will vary with the resistance of the muscle tissue between the electrodes. Typical values for the voltage are between 0.1 and 15 Volts, 0.2 and 12 Volts, 0.5 and 12 Volts, 0.5 and 10 Volts, or 0.5 and 7.5 Volts. In one embodiment, the voltage is 2.5 Volts, 5 Volts, 7.5 Volts or 10 Volts. Either a current source (voltage limited) or a voltage source (current limited) stimulator may be used.

It is also possible to use an asymmetric biphasic pulse, in which, for example, the first phase is shorter in duration than the second phase.

FIG. 5 shows an apparatus in accordance with an embodiment of the present invention, including a programmer unit 28 which may be utilised by a physician to set and adjust parameters of the implanted stimulator 10. The programmer unit 28 may include an appropriate means for communicating with the stimulator via transceiver 29, and may include a computing device. The control unit 19 is also arranged to transmit stimulator telemetry information indicative of one or more of the parameters of the stimulator 10, for detection by the programmer 28 via transceiver 29. The programmer unit 28 can therefore determine parameters of the stimulator from telemetry information and can adjust the parameters by transmitting control signals to the stimulator 10. The signal from the programmer may be able to selectively vary the output current, shape, frequency and/or pulse width or stimulation mode of the stimulation signal(s).

In operation, a physician adjusts parameters of the stimulation signal (s). The physician will note feedback from the patient as to the effect of the stimulus on bladder control, and may subsequently re-adjust the parameters until the stimulation is optimum. For example, patient perceived feedback may be used to set the maximum stimulation threshold of the smooth muscle sphincter (for example, any excess stimulation to the neosphincter may elicit and/or be perceived as an urgency event by the patient).

In the above-described embodiments, signals between the controller or programmer and the stimulator are RF signals. Other types of transmission media other than RF may be used. For example, microwave signals may be used for transmission, optical signals may be used, and in another embodiment magnetic transmission may be used.

Magnetic transmission may be used for the controller unit 12 to cause the stimulator to stop producing stimulation signals and therefore allow the patient to urinate. In this embodiment, the controller unit 12 may be a simple magnet which, when passed over a magnetic receiver of the stimulator 10, results in the stimulator ceasing to provide stimulation signals for contracting the sphincter.

As discussed above, the present invention is not limited to augmentation of smooth muscle sphincters for use in urinary incontinence. Any smooth muscle device for implantation in the body may be augmented in accordance with the present invention. Another application is a smooth muscle sphincter for treatment of fecal incontinence.

In the Australian provisional patent application referred above, Australian patent application number 2005905673, a treatment for fecal incontinence is proposed which involves stimulation of a smooth muscle sphincter wrapped about a portion of the anal canal or colo-rectal canal. In accordance with an embodiment of the present invention, the smooth muscle sphincter may be augmented.

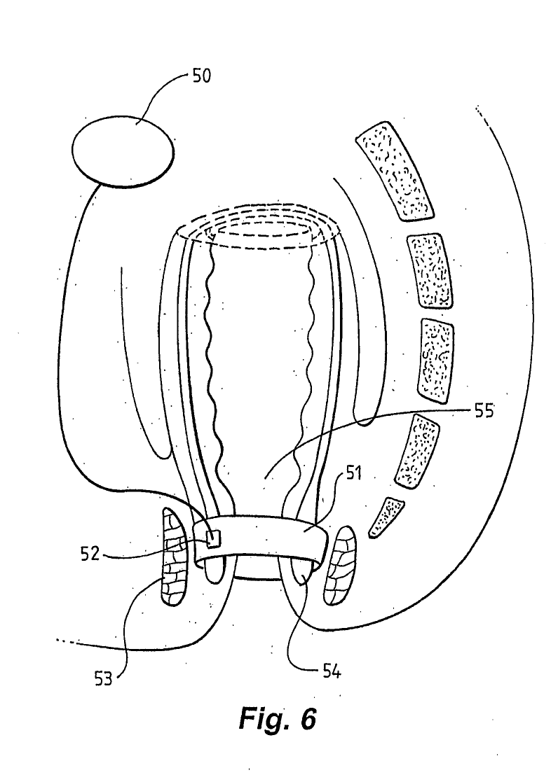

Referring to FIG. 6, a system and apparatus in accordance with an embodiment of the present invention, for treating fecal incontinence, are illustrated in schematic form. The system includes an apparatus comprising an implantable stimulator 50 and a device comprising smooth muscle tissue 51 which is arranged to be stimulated by a signal that is generated by the stimulator 50 and, in this embodiment, applied to the contractile tissue 51 via an electrode 52 conductively connected between the stimulator 50 and contractile tissue 52.

In this embodiment, the stimulator 50 may be of the same general construction as described above with reference to FIGS. 3, 4 and 5, with the signal characteristics being provided as appropriate for the fecal incontinence application.

The smooth muscle implant 51 in this embodiment is formed into a sphincter which is implanted about the anal sphincter region, in this embodiment proximate to the anus. In FIG. 6, the external anal sphincter is designated by reference numeral 53 and the internal anal sphincter by reference numeral 54. Failure of operation of the external and/or internal anal sphincters (perhaps because of nerve damage, or other reason) have lead to anal incontinence in this patient. Stimulation of the smooth muscle sphincter 51, in operation, causes it to contract and maintain closure of the anal-rectal canal 55, maintaining anal continence.

The stimulator 50 as discussed above includes a signal generator arranged to provide a stimulation signal for stimulating the smooth muscle sphincter 51. A lead 56 extends from the stimulator 50 to the electrode 52 at the smooth muscle sphincter 51, for providing the stimulation signal. The stimulation signal may be a signal of frequency and amplitude determined to maintain contraction of the smooth muscle sphincter 51 to facilitate fecal continence.

The stimulator 50 may also be arranged to produce a further electrical signal to stimulate the sphincter 51 to relax, to enable the patient to defecate. As an alternative to a further electrical signal, the stimulator 50 may be arranged to stop producing any electrical signal, and it is the absence of the signal that causes the sphincter 51 to relax. In this embodiment, the stimulator 50 is arranged to have the stimulation signal varied under control of the patient by way of an external controller.

The smooth muscle sphincter 51 is augmented in accordance with an embodiment of the present invention. The electrode 52 in this embodiment is arranged to release trophic and/or neurotrophic factors to promote growth of the smooth muscle sphincter 51. The smooth muscle sphincter 51 may include stem or proliferative muscle cells. Electrode 52 may mount a device with the trophic and/or neurotrophic factors or they may be impregnated within the electrode. Electrode embodiments incorporating such devices will be described in more detail later.

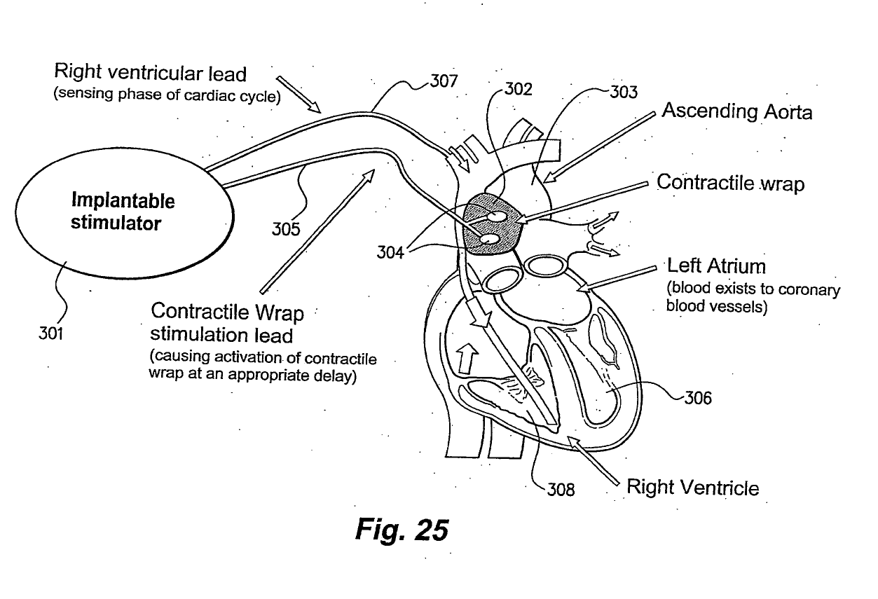

Referring to FIG. 25, a system and apparatus in accordance with an embodiment of the present invention, for treating a heart condition, are illustrated in schematic form.

The system includes an apparatus comprising an implantable stimulator 301, which is arranged to provide stimulation signals to contractile tissue 302, which is, in this example, placed as a wrap about the ascending aorta 303. Electrodes 304 at the contractile tissue 302 are conductively connected to the stimulator in order to transmit the signal from the stimulator 301 to the contractile tissue 302. A conductive lead 305 connects the stimulator 301 to the electrodes 304.

In operation, the contractile tissue 302 is caused to contract periodically to assist heart function. In this particular embodiment, the contractile wrap 302 is placed about the ascending aorta 303 before the blood flow reaches the coronary arteries. The stimulator 301 is timed to provide a signal to the contractile wrap 302 so that it contracts in order to provide a counter-pulsation effect during the latter phase of ejection of blood from the left ventricle 306. The contraction provides a “turbo-boost” increase in perfusion to the coronary blood vessels distal to the contractile wrap 304. It also has the beneficial effect of increasing blood flow peripherally by additional emptying of the ascending aorta 303.

In order to facilitate correct timing of the signal to the contractile wrap 302, a conductive lead 307 is connected between the right ventricle 308 and the implantable stimulator 301. This right ventricular lead 307 includes an electrode placed in the right ventricle 308 and provides signals back to the implantable stimulator 301 which includes a control unit which is able to determine from the sensor signals when electrical activation of the right ventricle 308 is occurring. At a predetermined delay after right ventricular 208 electrical activation, the stimulator provides the stimulation signal to the contractile wrap 302, to provide the counter-pulsation effect.

In this embodiment, the contractile tissue is smooth muscle tissue.

The contractile tissue wrap 302 is augmented in accordance with an embodiment of the present invention. The electrodes 304 may be arranged to release trophic and/or neurotrophic factors to promote growth. Stem cell proliferative muscle cells may be included. A device may be mounted for also release of trophic and/or neurotrophic practice.

The smooth muscle may be taken from anywhere or grown as discussed above for application with this invention. In one embodiment, the smooth muscle may be taken from the smooth muscle of the bladder and transplanted about the urethra, with its circulation in tact. Alternatively, the muscle is venous smooth muscle, anococcygeus smooth muscle or terminal ileum transplanted as a segment devoid of mucosa and having its circulation in tact. A further alternative is the dartos smooth muscle from the scrotum or a portion of the vagina or labia.

In addition, smooth muscle may be taken as a free graft. In this case, the tissue is separated from its normal circulation and becomes as vascularised by ingrowth of blood vessels at the side of implant.

The smooth muscle implant is not limited to a smooth muscle sphincter. Any smooth muscle implant operating in some other mechanical configuration to assist a bodily function may be augmented in accordance with the present of the invention. Any smooth muscle having a contractile function, for example, where it has effect on a tissue or organ in the body, may be augmented in accordance with an embodiment of the present invention.

In the above embodiments, power sources for the implantable stimulator will be provided in the form of batteries. These are not shown in the diagrams. The batteries may be replaceable or may be rechargeable via inductive recharging and are incorporated within the implantable stimulator.

As discussed above, the stimulator implant is preferably sealed and encased in a biologically inert material such as a bio-compatible silicone material. Metallic electrodes and leads are preferably of platinum-iridium alloy. The connecting wires are preferably insulated with a silicone coating. The implant is preferably placed between the abdominal muscle and the skin.

In the above embodiments, a single stimulation single signal generator is used to provide the electrical signal.

Other embodiments may use two or more signal generators. Other embodiments may use two or more stimulators, which may be placed in different locations.

In the above embodiments, electrodes are used to stimulate the smooth muscle implant. In PCT/AU2005/001698 referenced above, a suitable electrode arrangement is disclosed. A similar electrode arrangement may be used in accordance with the present invention as a device, or to mount a device, for delivering trophic and/or neurotrophic factor(s).

A description will be given firstly of an electrode in accordance with PCT/AU2005/001698 that may be used with the embodiments described above. Note that the invention is not limited to using this type of electrode, and other electrodes, such as button electrodes, may be utilised.

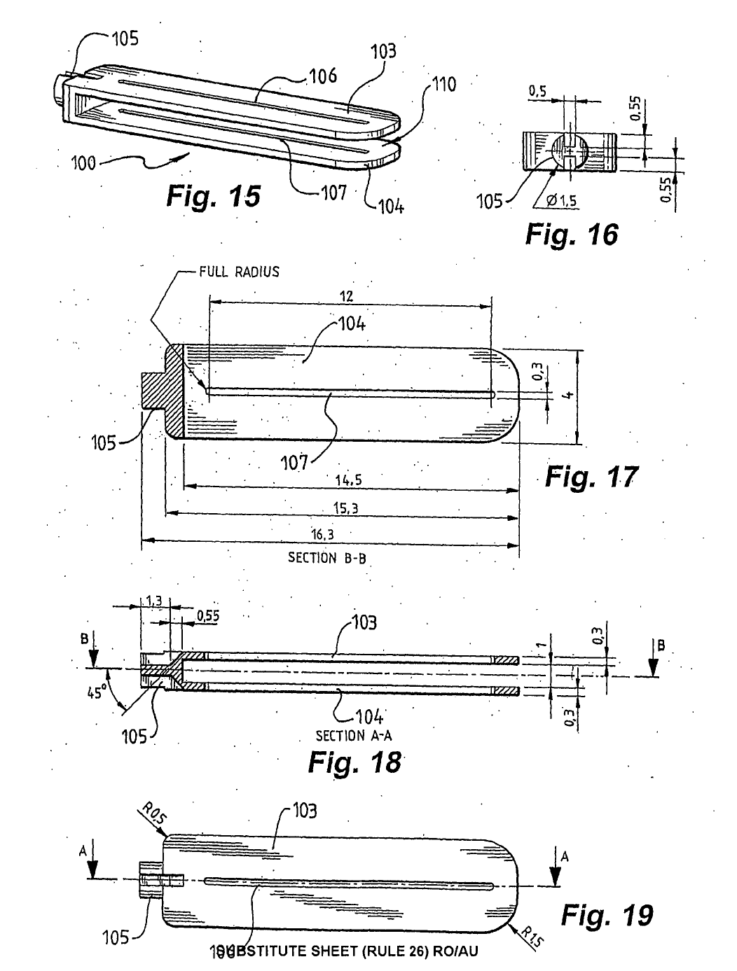

The electrode comprises a number of components. These include an electrode cover 100 (shown in most detail in FIGS. 14 through 18).

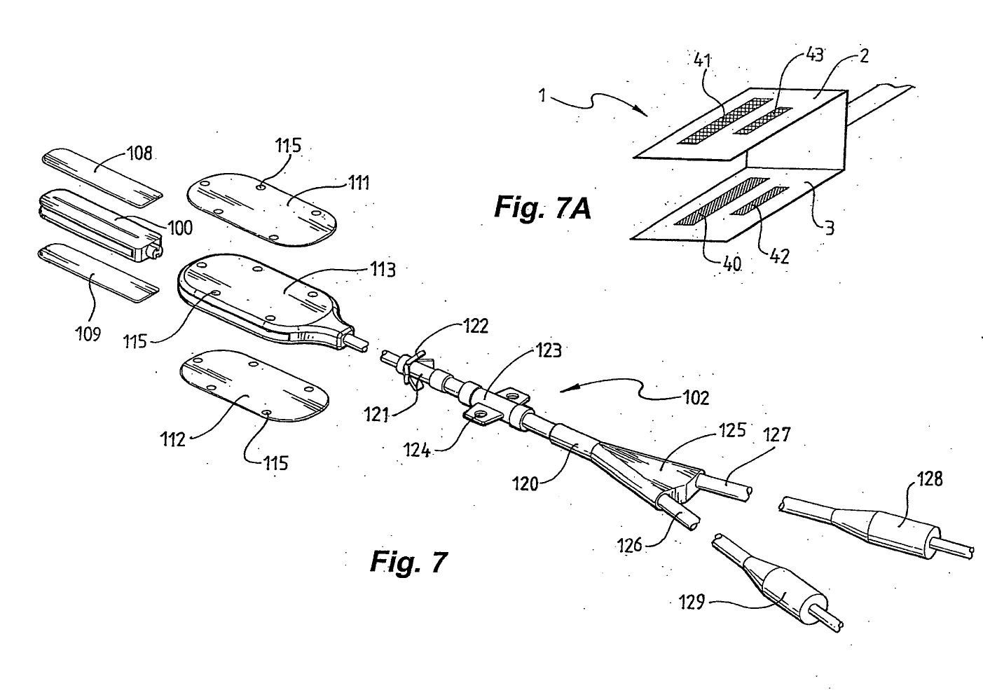

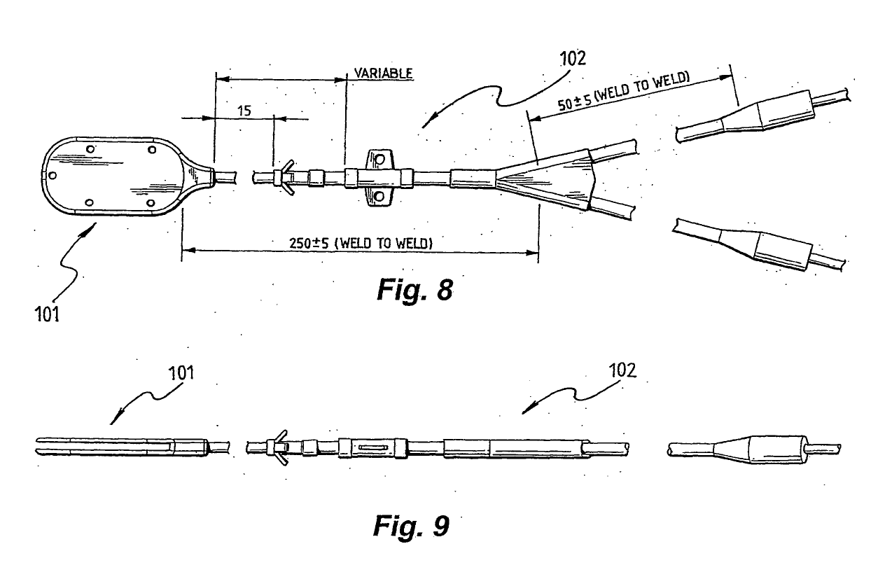

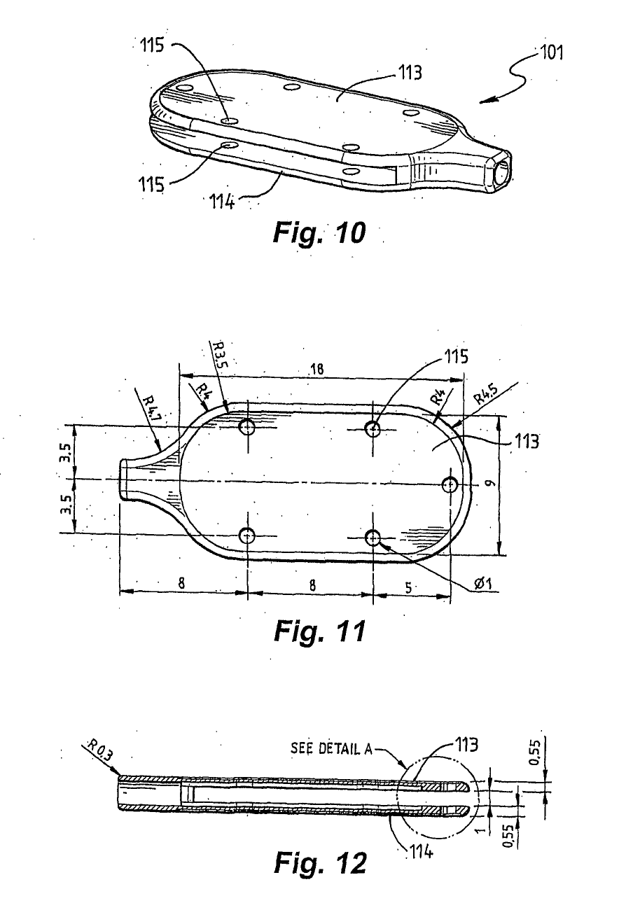

The components also include an electrode shroud (shown in best detail in FIGS. 10 through 13) and also an electrode lead 102 (shown in FIGS. 7, 8 & 9, together with the other components of the electrode arrangement).

In this embodiment first and second electrode elements are formed by the electrode cover 100, which includes insulating elements 103,104 extending from a base 105. The insulating extending elements 103,104 are formed with a slot 106,107, respectively, extending substantially along the length of the extending elements 103,104. When the electrode arrangement is assembled, platinum foil electrodes 108,109 (FIG. 7) are placed on the outer surfaces of the elements of the elements 103,104 so that they are insulated from the gap 110 formed between the elements 103,104 apart from the slots 106,107, which expose portions of the conductive plates 108,109 to the gap 110 (and, in use, to any tissue seated within the gap).

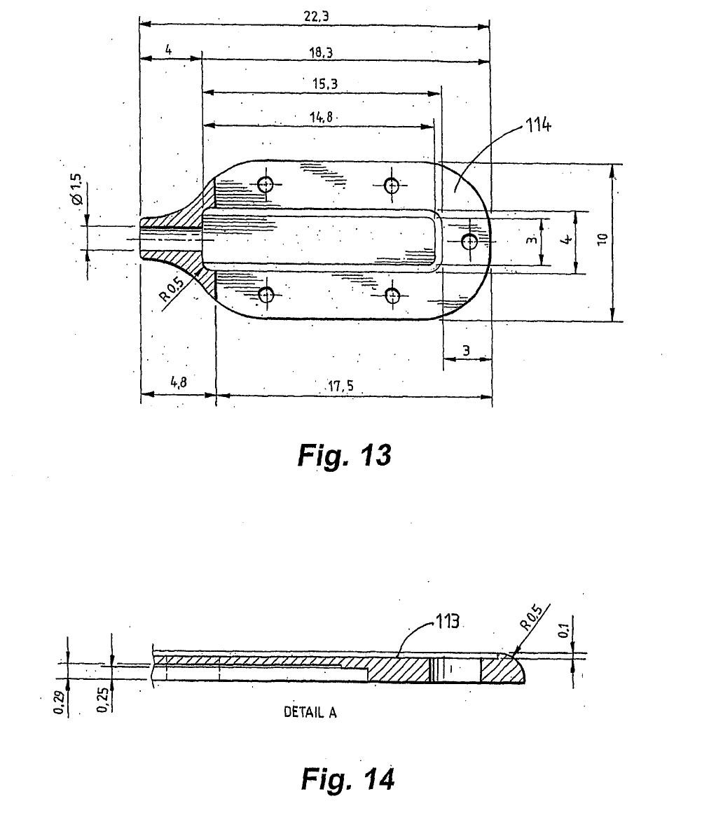

When assembled, the electrode cover 100 and platinum electrode foils 108,109 seat within the electrode shroud 101 as best shown in FIGS. 10, 11, 12 & 13. FIG. 13 in particular shown in cross-section where the electrode cover seats.

Electrode shroud 1 is formed from silicone. In order to provide reinforcement, PET mesh covers 111,112 are provided to fit to upper 113 and lower 114 extending portions of the shroud 101. Suture holes 115,116 are provided in the covers 111,112 and also in the elements 113,114 of the shroud 101. Note that the reinforcement can be provided by other means and is not limited to PET mesh. Further, the electrode shroud need not be in silicone but could be of other bio-compatible material and may not require re-inforcement. Further, note that other means for affixing to the tissue may be provided other than suture holes or instead of suture holes.

The electrode lead 102 is a multi-component arrangement which includes an outer insulating cover 120, a tine collar 121 including tines 122 for retaining the lead in position within a patient. It also includes a sutured collar 123 including suture holes 124 for suturing to patient tissue to also facilitate retaining the lead 102 in position. There is also bifurcation moulding 125 which enables the lead to split into two parts 126,127 which may contain separate conductors, and connectors 128,129 which may be arranged to contact to a simulation device.

In the above embodiments, the electrode arrangement includes a pair of electrode elements which extend away from a base which joins them together at their proximal ends. In a further embodiment, a single electrode element which is not joined at any base is provided. This single electrode element may be used to provide stimulation to contractile tissue on its own, or may be used together with one or more similar electrode elements to provide stimulation.

In the above described embodiments, each electrode element is provided with a single electrode. The single electrode is an elongate electrode extending substantially the majority of the length of the electrode element.

One advantage of having thin electrodes bounded by insulating material on either side is that the arrangement operates to confine the electric field produced by the electrode to the tissue immediately adjacent the electrode. This reduces or prevents stimulation of tissue that it is not desirable to stimulate eg. tissue external to a contractile tissue sphincter being controlled.

In operation, the electrodes 108, 109 and extending elements 103, 104 are positioned either side of a smooth muscle implant to enable signals to be transmitted to the implant for operation.

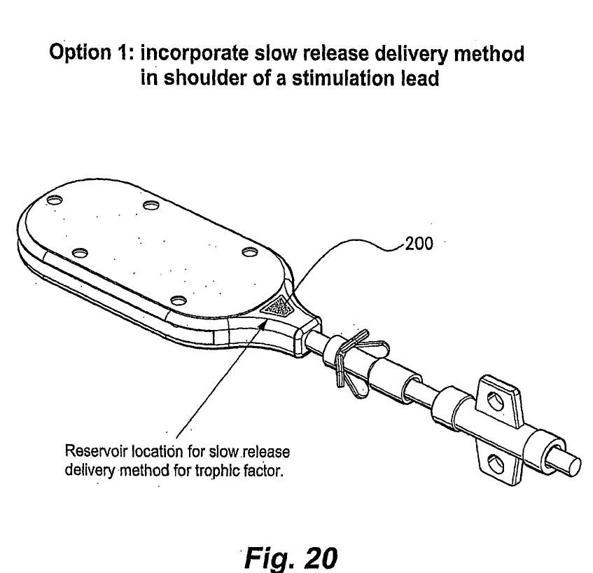

Electrodes with a similar structure to the electrode of FIGS. 7 through 19 may be used to provide delivery devices, or may operate as delivery devices, for delivering trophic or neurotrophic factors to a smooth muscle implant, in accordance with an embodiment of the present invention. Various embodiments are illustrated in FIGS. 20 through 24.

Referring to FIG. 20, a reservoir 200 in the shoulder of the electrode housing may contain trophic or neurotrophic factor.

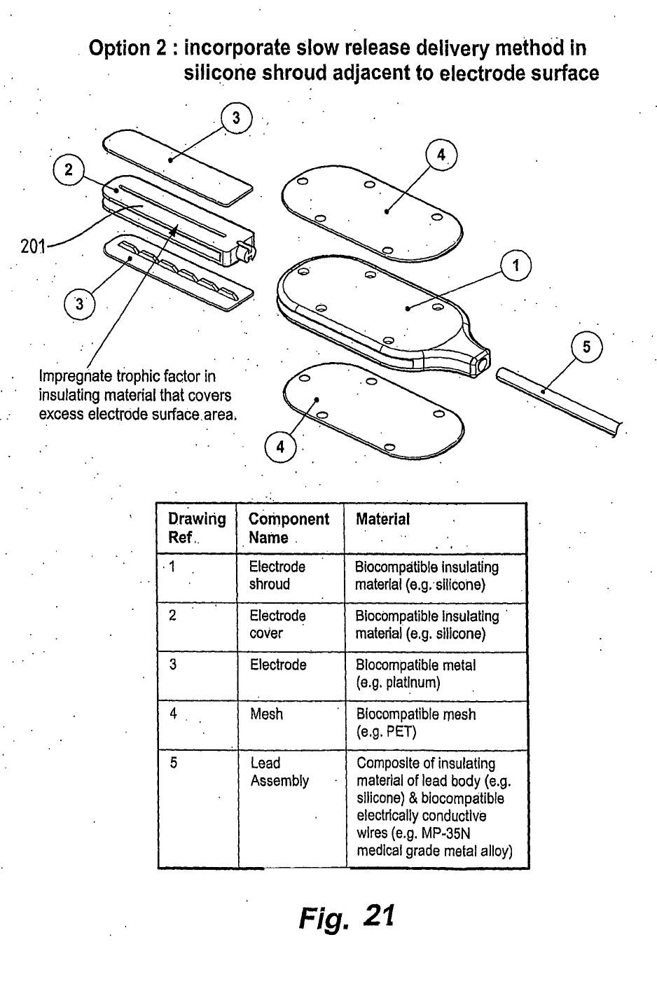

Referring to FIG. 21, the insulator 201 may contain impregnated trophic factor for release in use.

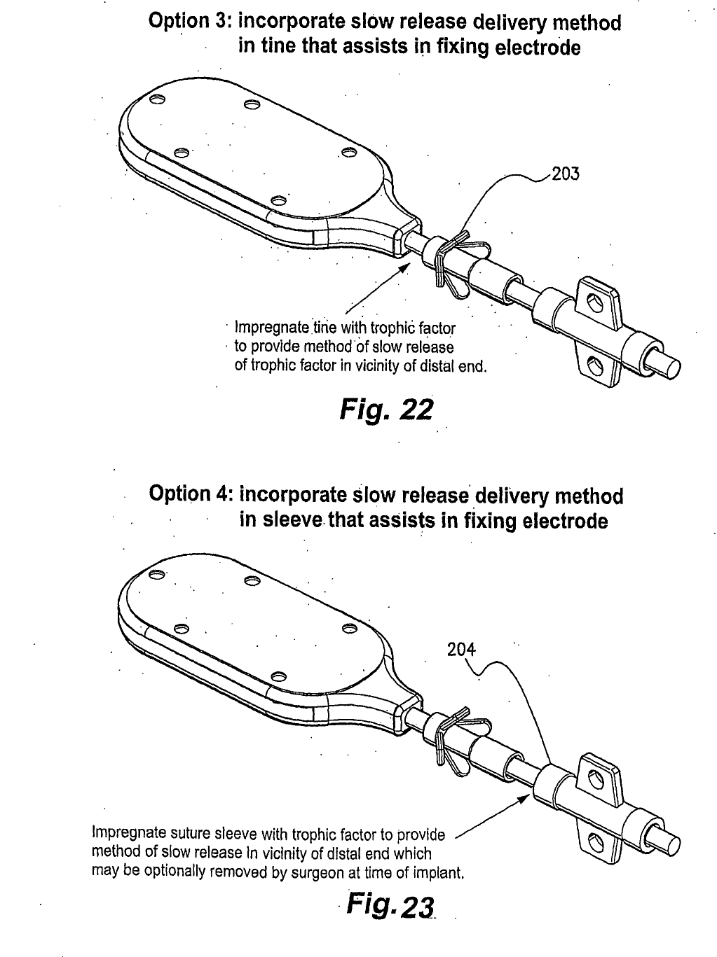

Referring to FIG. 22, the tines 203 that assist in affixing the electrode in place may incorporate trophic or neurotrophic factors for slow release.

Referring to FIG. 23, the suture sleeve 204 may incorporate trophic or neurotrophic factor for slow release.

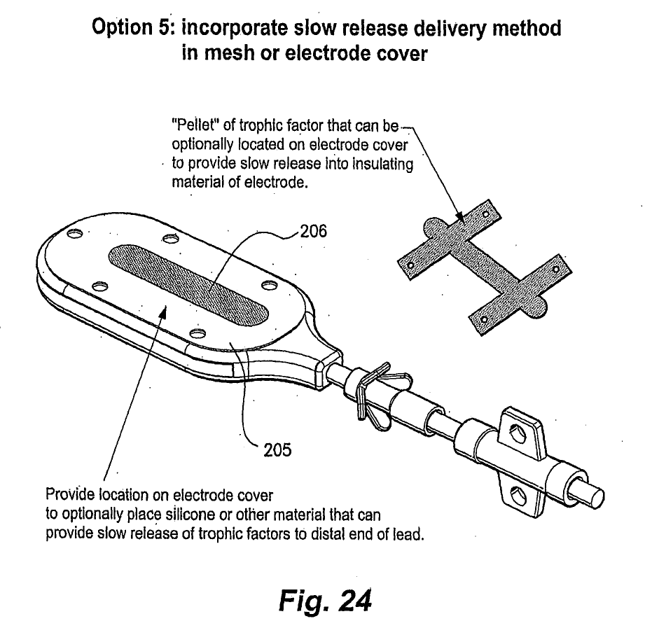

Referring to FIG. 24, a receptacle 205 may mount a silicone or other material device 206 and incorporates trophic or neurotrophic factors.

Alternative embodiments for introducing trophic or neurotrophic factors may include any of the following, either independently, or in combination where appropriate:

Incorporating a dispenser of the factor in the electrode lead. The factor would be contained within a cavity in the lead, held in a polymer carrier, or other suitable material;

Incorporating a reservoir for the factor in a distal portion of the lead and using a semi-permeable membrane, porous screen or mesh in conjunction with the reservoir to control the release of the factor;

Configuring the electrode to be porous, the distal end of the lead incorporating a polymer device containing the factor for release through the electrode;

Configuring the electrode to have a bore in which is placed a matrix, preferably of silicon, containing the factor. The electrode and matrix in this embodiment are configured to allow intrusion of local bodily fluid therein to distribute the factor about the electrode;

Configuring the electrode and/or the lead to include a portion which is a biocompatible solid material containing the factor within its pores for release thereabout when in place in the body; and

Configuring the electrode and/or the lead to include thereupon a biodegradable pad incorporating the factor for local release prior to or together with biodegradation of the pad.

As an alternative to utilising an electrode, a device may be placed separately for delivery of various trophic or neurotrophic factors. Further, a device may comprise a support for bodily tissue or organ, such as a mesh support, either impregnated or coated with trophic or neurotrophic factors for slow release or carrying devices which contain slow release trophic or neurotrophic factors. A non-limiting example of such mesh supports includes medical silicone rubber mesh supports such as those manufactured by NuSil Technology LLC (www.nusil.com). Alternatively, “nano-scale” devices may be used to deliver various trophic or neurotrophic factors. Such nano-scale delivery devices may include silicon devices with therapeutic substances encapsulated or absorbed therein, the silicon device or devices being within the body of the distal end of the stimulation lead.

Trophic factors and neurotrophic factors may also be delivered by a conductive coating on the electrode circuit itself (rather than impregnating electrode). This may provide a means of controlling the speed of release.

Growth factors may also be added to encourage revascularisation in smooth muscle. For example vascular endothelial growth factor (VEGF), or nerve growth factor (NGF) may be used.

Variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method of facilitating a smooth muscle implant, including the step of augmenting smooth muscle intended for an implant, or already implanted smooth muscle, in order to facilitate performance of the smooth muscle.

2. A method in accordance with claim 1, wherein the step of augmenting includes the step of adding proliferative smooth muscle cells to the smooth muscle.

3. A method in accordance with claim 1, wherein the step of augmenting includes the step of adding smooth muscle stem cells to the smooth muscle implant.

4. A method in accordance with claim 1, wherein the step of augmenting includes the step of adding trophic factor(s) in order to promote nerve and/or tissue growth.

5. A method in accordance with claim 4, wherein the step of adding growth factor includes adding trophic factor(s) arranged to stimulate growth of noradrenergic nerve fibers within a patient's anatomy into the smooth muscle.

6. A method in accordance with claim 4, wherein the step of adding trophic factor(s) is carried out by implantation of a slow release delivery mechanism into the patient's anatomy, after the smooth muscle has been implanted.

7. A method in accordance with claim 6, wherein the slow release delivery mechanism is associated with an electrode which is arranged to provide stimulation to the smooth muscle implant.

8. A method in accordance with claim 7, wherein the delivery mechanism is part of a housing mounting the electrode.

9. A method in accordance with claim 7, wherein the trophic factor(s) is impregnated into a part of the electrode for slow release.

10. A method in accordance with claim 6, wherein the delivery mechanism is associated with a supporting material in the patient's anatomy.

11. A method in accordance with claim 10, wherein the supporting material is a mesh supporting tissue or an organ.

12. A method in accordance with claim 10, wherein the mesh is impregnated with the trophic factor(s) for slow release.

13. A method in accordance with claim 11, wherein the device is supported by the support material, containing trophic factor(s) for slow release.

14. A method in accordance with claim 4, wherein the trophic factor is a growth factor for the growth of healthy smooth muscle tissue.

15. A method in accordance with claim 4, wherein the trophic factor(s) is a neurotrophic factor for the growth of healthy nerves.

16. A method in accordance with claim 1, wherein the step of augmenting includes the step of preparing a partial or an entire smooth muscle implant by tissue modelling.

17. A method in accordance with claim 1, wherein the smooth muscle implant is a sphincter.

18. A method in accordance with claim 17, wherein the smooth muscle implant is a neosphincter for treating urinary incontinence.

19. A method in accordance with claim 17, wherein the smooth muscle implant is a neosphincter for treating fecal incontinence.

20. A device consisting of smooth muscle, or primarily of smooth muscle, intended for implant or being implanted, to treat a medical condition, including smooth muscle augmented in accordance with a method of claim 1.

21. A device in accordance with claim 20, wherein the smooth muscle is intended or being a sphincter implant.

22. A device in accordance with claim 21, being intended as or being as a sphincter for treating urinary incontinence.

23. A device in accordance with claim 21, being intended or being a sphincter for treating fecal incontinence.

24. A system for managing a patient condition, comprising an implantable stimulator device and a smooth muscle device augmented in accordance with the method of claim 1.

25. A system in accordance with claim 24, wherein the augmented smooth muscle is a smooth muscle sphincter.

26. A system in accordance with claim 25, wherein the smooth muscle sphincter is a sphincter for the treatment of urinary incontinence.

27. A system in accordance with claim 25, wherein the smooth muscle sphincter is a sphincter for the treatment of a fecal incontinence.

28. A system in accordance with claim 35, wherein the smooth muscle sphincter is a sphincter for the treatment of esophageal reflux.

29. A system in accordance with claim 24, further comprising a controller which is operable by a user for controlling the implantable stimulator device.

30. A system in accordance with claim 24, further comprising a programmer, which is operable by a clinician to programme parameters of the implantable stimulator.

31. A system in accordance with claim 24, further comprising an implantable delivery device arranged to deliver neurotrophic and/or trophic factor(s) to promote nerve growth and/or tissue growth in the smooth muscle implant.

32. A system in accordance with claim 31, wherein the implantable delivery device is mounted by an electrode arranged to apply a signal from the stimulator to the smooth muscle implant.

33. A system in accordance with claim 32, wherein the delivery device is part of the electrode.

34. A system in accordance with claim 33, wherein component(s) of the electrode are impregnated with trophic and/or trophic factor(s).

35. A system in accordance with claim 30, wherein the delivery device is mounted by supporting material placable in the vicinity of the smooth muscle implant.

36. A system in accordance with claim 35, wherein the trophic and/or neurotrophic factor is impregnated into the supporting material.

37. A system in accordance with claim 36, wherein the supporting material is a supporting mesh.

38-46. (canceled)
